(19) United States
(12) Patent Application Publication
(10) Pub. No.: US 2013/0245496 A1
(43) Pub. Date: Sep. 19, 2013

(54) URINARY CATHETER ANTI-REFLUX AND PATHOGEN BLOCK DEVICE

(71) Applicants: Mark Edward Wells, Athens, AL (US); Patricia Strader, Greensburg, KY (US)

(72) Inventors: Mark Edward Wells, Athens, AL (US); Patricia Strader, Greensburg, KY (US)

(21) Appl. No.: 13/737,769

(22) Filed: Jan. 9, 2013

Related U.S. Application Data
(60) Provisional application No. 61/631,644, filed on Jan. 9, 2012.

(51) Int. Cl. A61M 25/00; A61B 10/00

(52) U.S. Cl. CPC A61M 25/0017; A61B 10/007
USPC 600/581; 604/544; 604/328

(57) ABSTRACT

The present disclosure generally pertains to a device and system for preventing urinary backflow into the urinary tract of a patient. The device comprises a one-way valve which allows for unobstructed urine flow in the direction of the urine towards the urine collection container and away from the catheter. The device prevents backflow into the catheter in situations where the urine collection container is raised above the level of the bladder. The components on the exit side of the valve are coated with an antimicrobial substance to prevent bacterial contamination of the system. The exit volume also contains a silver coated high surface area element to serve as an antibacterial kill site. The inlet side of the device has a volume and access port for urine sampling, with a one-way valve element providing isolation from contamination from the urine collection system.

### CROSS REFERENCE TO RELATED APPLICATION

This application claims priority to U.S. Provisional Patent Application No. 61/631,644, entitled “Urinary Catheter Anti-Reflux and Pathogen Block Device,” and filed on Jan. 9, 2012, which is incorporated herein by reference.

### RELATED ART

Urinary tract infections (“UTIs”) are the most common type of healthcare-associated infection, accounting for more than 30% of infections reported by acute-care hospitals. Virtually all healthcare-associated UTIs are caused by use of instrumentation in the urinary tract. Catheter-associated urinary tract infections (CAUTIs) are associated with increased morbidity, hospital costs, and length of stay. In addition, bacteriuria, the presence of bacteria in urine not due to contamination from urine sample collection, commonly leads to unnecessary antimicrobial treatments as urinary drainage systems are often reservoirs for multi-drug resistant bacteria. An estimated 17% to 69% of CAUTIs may be preventable with recommended infection control measures, leading to the prevention of 380,000 infections and 9000 deaths related to CAUTI per year.

The most common cause of UTIs is the backflow of urine into the urinary tract when the catheter collection container is raised above the bladder level. Microbial pathogens can enter the urinary tract by migration along the outside of the catheter in the periurethral mucous sheath or by movement along the internal lumen of the catheter from a contaminated collection container or the catheter-drainage tube junction. Reductions in the numbers of such CAUTIs will have a positive effect on patient health and will decrease the overall cost of treating patients utilizing urinary catheters.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1A is a diagram illustrating an exemplary embodiment of an anti-reflux device.

FIG. 1B is a side view illustrating an exemplary embodiment of an anti-reflux device.

FIG. 2 is a diagram illustrating an exemplary embodiment of a hose barb connection, such as depicted by FIG. 1.

FIG. 3 is a diagram illustrating an exemplary embodiment of a duckbill backflow prevention valve.

FIG. 4 is a diagram illustrating an exemplary embodiment of a cruciform backflow prevention valve.

FIG. 5 is a diagram illustrating an exemplary embodiment of an exit device, such as depicted by FIG. 1.

FIG. 6 is a cross sectional view of an exemplary embodiment of an exit device, as depicted in FIG. 5 taken at section line 6-6.

FIG. 7 is a diagram illustrating an exemplary embodiment of a catheter anti-reflux system.

### DETAILED DESCRIPTION

This presently disclosed devices and systems prevent the backflow of urine into a catheter from both the catheter interconnection tube and the urine collection container through the use of a one-way or check valve. In certain embodiments, the device contains an anti-microbial agent that is compatible with the human body. The device provides a valve with a low cracking pressure, or pressure required to open the valve, to allow passage of urine. The device also prevents blockage of the valve caused by the presence of solids in the urine stream. Very low opening pressure is required to prevent migration of urine back into the urinary tract. The valve and other device components do not act as a bacteria source and may be sanitized using methods known in the art, for instance by autoclave, gas sterilization or irradiation methods.

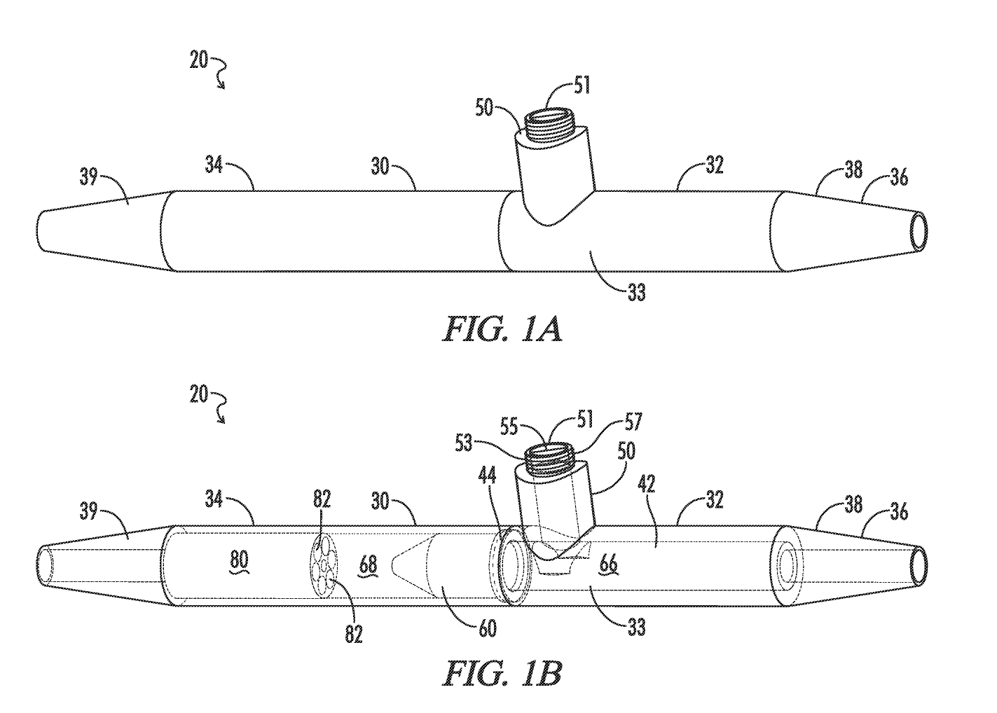

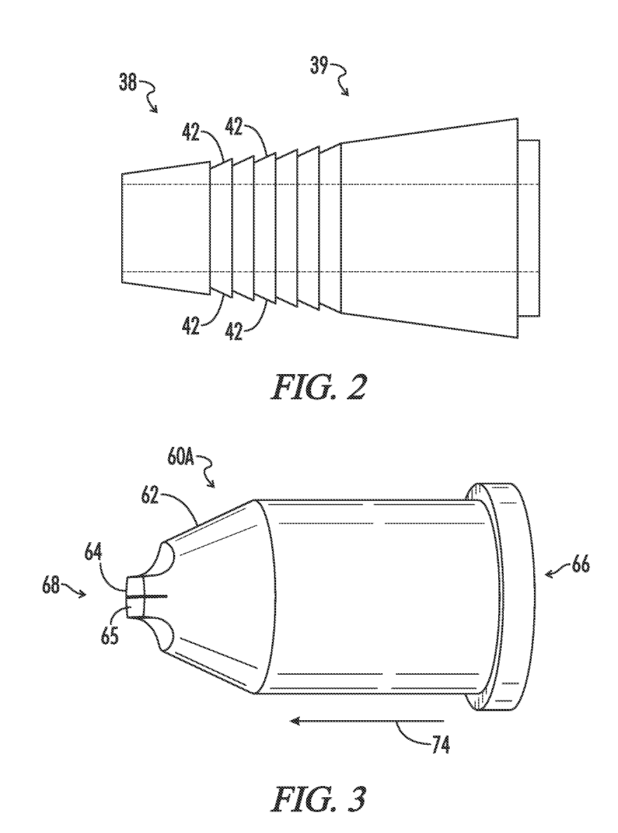

FIG. 1A is a diagram illustrating an embodiment of an anti-backflow catheter device 20. The device 20 comprises an elongated hollow tubular member 30 having an inlet chamber 32 and an outlet chamber 34. Inlet chamber 32 further comprises a tapered end portion 36 which functions as a catheter connection. Urine enters the device 20 through the tapered end portion 36, enters inlet chamber 32 and flows towards a medial portion 33 of the tubular member 30. Tubular member 30 incorporates standard hose barb connections 38 and 39 placed at the outside ends of the inlet chamber 32 and outlet chamber 34, respectively. The connections 38 and 39 serve as attachment points for the catheter end (38) and urine collection end (39) tubing. A hose barb connection is generally a tapered device with one or more continuous ridges or bumps on a fitting that are used to grip the inside diameter of a tube and seal the connection. An exemplary hose barb connection is illustrated in FIG. 2. Connections 38 and 39 are tapered cylindrical pieces or parts located at the at the outside ends of the inlet chamber 32 and outlet chamber 34 for attaching and securing tubing leading to the catheter or urine collection container (not shown). Each barb 38 and 39 comprises one or more of evenly spaced rings 43 with increasingly large diameters. The widest portion of each ring is formed with an angular barb-like protrusion 42. The tubing leading to the catheter and urine collection container (not shown) is easily installed on connections 38 and 39 because there is little resistance when fitting the tubing over the rings 43 in the direction towards a medial portion 33 of tubular member 30. The tubing is flexible and expands over the barbs 42 after placement over the connections 38 and 39. Grip and seal occur as the tube begins to relax to its original inside diameter behind the barbs 42 and thus resists any force applied to remove the tubing. Barb-like protrusions grip the inside surfaces of the tubing and resist forces in the direction away from a medial portion 33 of tubular member 30. The hoses therefore snugly fit over connections 38 and 39 and prevent slipping or leaking of fluids. Connections 38 and 39 help to prevent twisting or kinking of the catheter and urine collection container tubing (not shown). In one embodiment, inlet hose barb connection 38 may be coated on its exterior with at least one layer of an antimicrobial material to prevent microbial growth at the catheter connection.

Referring again to FIG. 1A, the tubular member 30 is attached directly to the catheter (not shown) via hose barb connection 38 to minimize the volume of urine which may backflow into the bladder. The tubular member 30 is constructed of a rigid, high-strength and transparent material which retains its form over a wide range of temperatures. One example of such a material is polysulfone, which belongs to a family of thermoplastic polymers. These polymers are known for their toughness and stability at high temperatures and will not promote microbial growth. This material may be sanitized using methods known in the art, for instance by autoclave, gas sterilization or irradiation methods.

Turning now to FIG. 1B, a needleless sample valve 50 is placed at the medial portion 33 of tubular member 30. In one embodiment, the sample valve 50 is constructed from molded polycarbonate materials and permanently attached to the tubular member 30. Sample valve 50 allows access to a sample volume 52 of urine collected directly from the catheter (not shown). This allows for aspiration of a small amount of urine, for example 10 cc, for further examination (i.e., urinalysis or culture). Access to the sample volume 52 is possible without the use of a needle or the need to permanently puncture the tubular member 30. In one embodiment, the sample valve 50 is sealed with a molded silicon rubber plug 51. The plug 51 comprises a slit 55 which travels its length. In one exemplary embodiment, a syringe with a Luer lock tip (not show) may be mated with sample valve 50. A standard syringe Leur lock tip (not shown) generally comprises a female threaded end surrounding a plastic tapered tip. In one exemplary embodiment, the syringe female threaded end is twisted onto the threaded sample valve 50. The plastic threaded tip fits between the slit 55 and then enters sample volume 52. The slit returns to the closed position when the syringe is twisted off the sample valve 50 (i.e., after withdrawal of a urine sample) and the tapered tip is removed.

Referring again to FIG. 1B, the inlet chamber 32 and outlet chamber 34 are separated by valve element 60. Valve element 60 serves as a backflow prevention device and blocks the movement of possibly contaminated urine from the collection container back into the catheter.

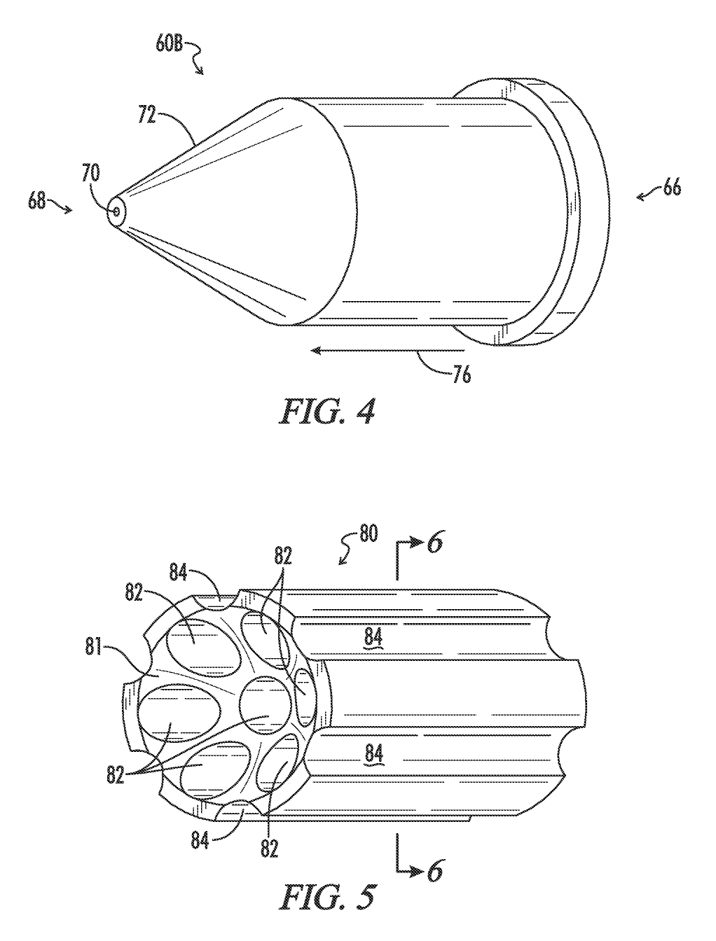

FIGS. 3 and 4 illustrate two exemplary embodiments of the of the valve element 60. As shown in FIG. 3, valve element 60A is in the form of a truncated cone 62 incorporating a slit 64 in the valve face 65. This type of valve element is often referred to as a “duckbill” valve. The valve 64B illustrated in FIG. 4 utilizes a “cruciform” shaped geometry incorporating a slit 70 through the tip of a cone-shaped element 72. Valves 60A and 60B open under positive pressure in the desired flow direction (indicated by arrows 74 and 76). At negative pressure differentials, valve elements 60A and 60B form positive seals at the slit locations 64 and 70, thus preventing backflow of urine in the directions opposite of arrows 74 and 76 (i.e., towards the catheter tubing). The valves 60A and 60B are constructed of flexible, elastomeric materials such as, for example, silicone or fluoro-silicone elastomer. These materials are biocompatible and will not cause adverse reactions in the body. Valve elements 60A and 60B have low cracking (opening) pressures, for instance pressure less than about 0.03 psi. Thus, urine flow from the catheter tubing (not shown) through the inlet side 66 of the valve elements 60A and 60B is not impeded during normal urine production, maintaining unrestricted urine flow. The valves 60A and 60B are designed to prevent entrapment of solids as they pass to the collection container. Duckbill valve 64A will pass solid objects, for instance as large as about 1.5 mm through slit 64. In addition, valves 60A and 60B are designed to pass viscous liquids, for example up to about 3000 centipoise viscosity. The cruciform geometry embodied by valve 60B (FIG. 4) allows the passage of urine flow with a higher or larger solid content, for example solid objects with an equivalent diameter of one-half the interconnecting tube diameter, or approximately 3 mm.

The exemplary catheter valves 60A and 60B prevent backflow of potentially contaminated urine into the catheter in the event that the urine collection container is not maintained a height below the level of the bladder. The valves 60A and 60B will prevent backflow at pressure heads in excess of the height of the urine collection container. For example, the urine collection container may be lifted to a maximum of 8 ft. tubing above the catheter without backflow or reflux.

In an additional embodiment, the interior surfaces of tubular member 30 at the exit side 68 of valve 60 may be coated with at least one thin continuous layer of a substance with the ability to reduce the incidence of bacterial growth. In one embodiment, the interior surfaces of outlet chamber 34 at the exit side 68 of valve 60 and the internal and external surfaces of valve 60 are covered in at least one layer of a silver or silver alloy compounds. Other suitable materials may include platinum or other metals. Silver ions and silver alloy compounds show a toxic effect on some bacteria, viruses, algae and fungi, interfering with the reproduction process of the pathogen without toxicity to humans. The chemical properties of silver in the ionized form (Ag⁺) produce the antimicrobial properties. Catheters utilizing silver alloy coatings are more effective than non-coated catheters for reducing bacteriuria in adults having short-term catheterization in hospitals. The valve will serve as a kill site for any reflux containing microbial material.

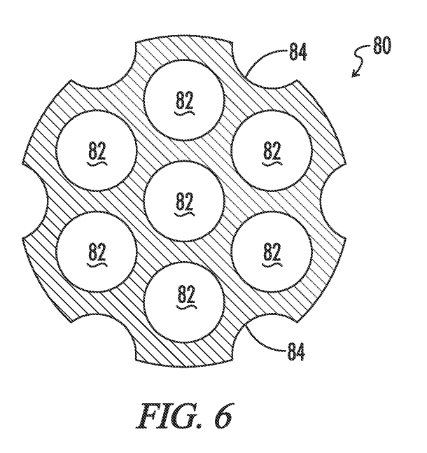

In an additional embodiment, the anti-backflow device 20 disclosed herewith further comprises an antimicrobial exit device 80. In one embodiment as is illustrated in FIG. 5, the exit device 80 is constructed as a cylindrical polycarbonate cartridge containing a plurality of hollow internal passages 82 and external grooves 84 traveling its length. Unlike the exit device illustrated in FIG. 1A, the exit device 80 illustrated in FIG. 5 comprises a concave face 81 and exterior grooves 84. This configuration of face 81 and grooves 84 increases the antimicrobial surface area available for interaction with the urine stream. Passages 82 and grooves 84 are also illustrated in FIG. 6, which shows a cross sectional view of the exit device 80. The exit device 80 resides within the outlet chamber portion 34 of the tubular member 30. It is to be understood that other embodiments of the exit device may exist. For instance, the outlet chamber portion 34 of the tubular member 30 may be filled with silver coated spheres. In one embodiment, the surfaces of exit device 80, internal passages 82 and external grooves 84 are coated with a thin layer of an antimicrobial substance, such as a silver compound. As described above, the silver confers antimicrobial properties to the exit device due to the release of silver ions. The channels 82 and grooves 84 increase the surface area of the silver coated surfaces, allowing greater exposure of potentially contaminated urine to the antimicrobial silver coating. In addition, the large number of passages 82 and external grooves 84 allow for the steady flow of urine through the exit device 80 and prevents blockages caused by solids normally found in urine. Placement of the exit device 80 in the outlet chamber 34 acts to (1) kill residual bacteria in the urine travelling through the device from the catheter, and (2) eliminate bacteria from any urine reflux backflow from the urine collection container. Movement of urine into or out of the outlet chamber 34 must occur through the exit device 80.

The anti-backflow device 20, valve 60 and other devices described herein may be made by conventional methods as known by one of skill in the art. In one embodiment, the components are assembled using ultrasonic bonding, which leaves no residual bonding material. The antimicrobial coatings presently disclosed may be made by conventional methods as known in the art. In order to avoid the introduction of any microbes into device 20, the device 20 and associated catheter and tubing may be sanitized using methods known in the art, for instance by autoclave, gas sterilization or irradiation methods.

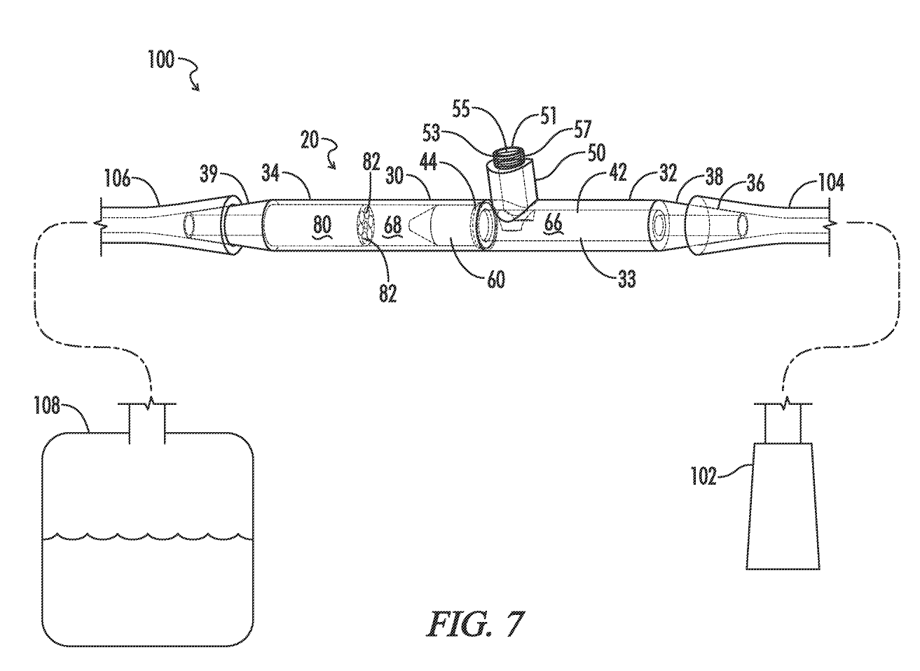

FIG. 7 shows one embodiment of a catheter anti-reflux system 100 as presently disclosed. A urinary catheter 102 is utilized to assist in the removal of urine from a patient. The catheter 102 is connected to the device 20 inlet chamber 32 via rubber catheter tubing 104. As discussed in detail previously, catheter tubing 104 is secured to the device via hose barb connection 38. Device 20 prevents backflow of urine back into the catheter through the use of one-way valve 60. In addition, the device 20 provides antimicrobial surfaces on the interior of the outlet chamber 34 as well on a large surface area exit device 80. These structures function to kill any residual or refluxed bacteria. Finally, device 20 provides a urine sample volume 66 which may accessed from a needleless sample valve 50, both or which are protected from backflow contamination. Urine collection tubing 106 is attached to the outlet chamber 34 by hose barb connection 39. The urine collection tubing 106 leads to a urine collection container 108.

In operation, the pressure of the urine flow emanating from a patient directs the urine through the catheter tubing 104 where it enters the anti-reflux device 20 at the inlet end portion 36. The device 20 and the catheter tubing 104 are connected via hose barb connection 38, thus preventing leakage. The exterior surfaces of hose barb connection 38 may be coated with an antimicrobial material to prevent contamination from occurring at this interface. The urine flow continues into the inlet chamber 32 of tubular member 30, filling the sample volume 42. A sample of fresh urine may be collected through the needless sample valve 50 by opening the non-puncture mechanism 51 and removing the urine. Sampling on the inlet side 66 of the valve element 60 prevents backflow of contaminated urine from the urine collection container and allows the sample to be representative of the true bladder output. The urine flows over the interior surfaces of the valve element 60 and reaches the outlet side 68 of the valve 60. The cracking or opening pressure of the valve 60 is low, for example less than 0.03 psi, allowing the outlet side 68 of valve element 60 to open under normal urine flow pressure.

The urine flow travels through the outlet side 68 of valve element 60 and interacts with the antimicrobial coated interior surfaces of the outlet chamber 34, which functions to kill any residual bacterial in the urine. The construction of the one-way valve element 60 prevents its opening and backflow of urine flow into the valve element 60, the medial portion 33 of the tubular member 30 and into the catheter (not shown) at head pressures of up to, for example, 10 feet of water. The continuing pressure of the urine flow pushes the urine stream through the antimicrobial-coated passages 82 and external grooves 84 of exit device 80. The large surface area provided by passages 82 and external grooves 84 ensure exposure of the entire urine stream to the antimicrobial coating. The urine flow then continues out of outlet chamber 34 of device 20 and into the tubing connecting the device 20 with the urine collection container 108. The urine collection container tubing 106 and the device 20 are attached via hose barb connection 39, thus preventing leakage. Any subsequent reflux of contaminated urine from the urine collection container 108 into the outlet chamber 34 will travel through the exit device 80 and will be exposed to the antimicrobial coated surfaces of passages 82 and external grooves 84. The presently disclosed device and system prevents reflux from traveling the urine collection system back to the bladder through the use of a one-way check valve, provides an antimicrobial “kill” area for bacteria and other pathogens at the entrance to the catheter, and provides a sampling volume adjacent to the collection catheter which is isolated form the urine collection system.

Now, therefore, the following is claimed:

1. A catheter reflux prevention device, comprising
a tubular member having an inlet for receiving urine from tubing connecting the tubular member to a catheter, the tubular member having an outlet for permitting the urine to egress from the tubular member, the tubular member defining an inlet chamber and an outlet chamber; and
a one-way valve defining the boundary between the inlet chamber and the outlet chamber.

2. The device of claim 1, further comprising a cartridge positioned within the outlet chamber and defining a passage for the urine, wherein a surface of the cylindrical cartridge defining the passage is coated with at least one layer of an antimicrobial substance.

3. The device of claim 2, wherein the antimicrobial substance comprises silver.

4. The device of claim 2, wherein an interior surface of the outlet chamber is coated with at least one layer of an antimicrobial substance.

5. The device of claim 1, further comprising a sample valve having an opening through which samples may be drawn from the inlet chamber.

6. The device of claim 1, further comprising a needless sample valve having an opening through which samples may be drawn from the inlet chamber.

7. The device of claim 1, wherein the one-way valve is a duckbill valve.

8. The device of claim 1, wherein the one-way valve is a cruciform valve.

9. The device of claim 1, further comprising barbed connections located at an outside ends of the inlet.

10. A system for preventing catheter reflux, comprising:
a catheter;
a catheter reflux prevention device coupled to the catheter via tubing for passing urine from the catheter to the catheter reflux prevention device, the catheter reflux prevention device having a tubular member and a one-way valve positioned within the tubular member between an inlet chamber and an outlet chamber of the tubular member; and
a urine collection container coupled to the catheter reflux prevention device via tubing for passing the urine from the outlet chamber to the urine collection container.

11. The system of claim 10, further comprising a cartridge positioned within the outlet chamber, wherein a surface of the cartridge is coated with at least one layer of an antimicrobial substance.

12. The system of claim 11, wherein the antimicrobial substance comprises silver.

13. The system of claim 11, wherein the cartridge has at least one hole for passing the urine through the cartridge.

14. The system of claim 10, wherein the catheter reflux prevention device has a sample valve for drawing samples from the inlet chamber.

15. The device of claim 10, wherein the catheter reflux prevention device has a needleless sample valve for drawing samples from the inlet chamber.

16. The device of claim 10, wherein the catheter reflux prevention device has barbed connections located at outside ends of the inlet and outlet chambers.

17. A method for preventing urinary catheter reflux, the method comprising:
passing urine from a catheter through tubing to a catheter reflux prevention device that is coupled between the catheter and a urine collection container;
passing the urine through a one-way valve within the catheter reflux prevention device; and
passing the urine from the one-way valve to the urine collection container.

18. The method of claim 17, further comprising passing the urine from the one-way valve through a cartridge positioned in the catheter reflux prevention device, wherein a surface of the cartridge is coated with at least one layer of an antimicrobial substance.

19. The method of claim 18, wherein the antimicrobial substance comprises silver.

20. The method of claim 17, further comprising removing a urine sample from the inlet chamber through a sample valve of the catheter reflux prevention device.

21. The method of claim 17, further comprising removing a urine sample from the inlet chamber through a needleless sample valve of the catheter reflux prevention device.

22. The method of claim 17, wherein the one-way valve is a duckbill valve.

23. The method of claim 17, wherein the one-way valve is a cruciform valve.
